(19) United States
(12) Patent Application Publication
(10) Pub. No.: US 2020/0245939 A1
(43) Pub. Date: Aug. 6, 2020

(54) ELECTRONICS MODULE FOR A WEARABLE SENSOR SYSTEM AND WEARABLE SENSOR SYSTEM

(71) Applicant: WORKAROUND GMBH, Munich (DE)

(72) Inventors: Hans SITTIG, Munich (DE); Sean ENGINEER, Munich (DE)

(21) Appl. No.: 16/781,734

(22) Filed: Feb. 4, 2020

(30) Foreign Application Priority Data
Feb. 4, 2019 (DE) 10 2019 102 730.6

(51) Int. Cl. A61B 5/00; H05K 3/46

(52) U.S. Cl. CPC A61B 5/6825; A61B 5/7495; H05K 3/4691

(57) ABSTRACT

An electronics module for a wearable sensor system has a housing that includes an outer wall, at least one contact element, and a carrier for the at least one contact element. The contact element extends through the at least one outer wall and is attached to the carrier, the carrier including at least one electrical conductor for electrical connection of the contact element and being designed to be flexible. Furthermore, a wearable sensor system is shown.

### FIELD OF THE DISCLOSURE

The disclosure relates to an electronics module for a wearable sensor system and a wearable sensor system.

### BACKGROUND

Wearable sensor systems are known and are used to make sure that in mixed activities that require both the user to have his/her hands free and the use of sensors, the appropriate sensor is always carried on the user's body. This allows the working speed of this activity to be significantly improved.

However, the electronics modules, i.e. modules in which the actual sensor is arranged, are usually very large, which greatly impairs the user when using his/her hands during the activity. For example, in tasks with limited space available, the user will frequently bump on something or find the sensor system annoying.

### SUMMARY

Thus, there is a need to provide an electronics module for a wearable sensor system as well as a wearable sensor system which are only small in size.

The object is achieved by an electronics module for a wearable sensor system, having a housing that includes an outer wall, at least one contact element, and a carrier for the at least one contact element. The contact element extends through the at least one outer wall and is attached to the carrier, the carrier including at least one electrical conductor for electrical connection of the contact element and being designed to be flexible.

Due to the fact that the carrier is constructed to be flexible, the carrier is especially thin so that the overall height of the electronics module and therefore of the entire sensor system can be reduced.

In the context of this disclosure, flexible means in particular that the carrier alone permits a movement of the contact elements into the interior of the housing perpendicular to the outer wall or cannot reliably and permanently fix the contact elements in place. A flexible carrier is given, for example, if its thickness is less than 1 mm.

For example, the carrier is a rigid-flex printed circuit board, as a result of which the carrier can be implemented cost-effectively.

Rigid-flex printed circuit boards may have sections of different flexibilities. More particularly, to this end, flexible layers may be attached to each other and/or on top of each other.

In one configuration of the disclosure, the carrier includes at least one line section and a holding section, the at least one contact element being attached to the holding section. In this way, the carrier can reliably perform various functions.

The line section and the holding section are constructed together in one piece.

The contact elements may be attached to the carrier by gluing, for example by means of adhesive pads. Alternatively or additionally, the attachment may be realized by means of an interlocking fit, for instance by a plug connection.

In order to reliably attach the contact elements to the carrier, the holding section may be formed at least partly from an FR-4 material and/or the carrier may include a reinforcement layer in at least parts of the holding section.

The reinforcement layer is, for example, also made from an FR-4 material.

In one embodiment of the disclosure, the thickness of the carrier, in particular the thickness of the holding section of the carrier, is equal to or less than 0.8 mm, in particular equal to or less than 0.6 mm. This allows the overall height of the electronics module to be further reduced.

The line section may be thinner than the holding section.

In one design variant, the electronics module includes at least one sensor, in particular a camera, and/or a main board, so that the electronics module is an independent sensor unit.

The sensor is more particularly a bar code scanner with a camera. In the context of this disclosure, one-dimensional, multidimensional, two-colored and/or multi-colored codes fall within the term “bar code”, and the bar code scanner is adapted to detect one or all of these types of bar codes.

It is also conceivable that the sensor is an RFID reader.

In order to further reduce the overall size of the electronics module, the main board may be arranged perpendicular to the outer wall and/or the carrier may be attached to the main board.

For example, the carrier establishes an electrical connection between the main board and the at least one contact element.

To protect the sensor, the sensor may be provided at least partly inside the housing and/or may be electrically connected to the main board.

In order to reliably fix the contact element in place, in particular in relation to the opposing wall or the cover, the electronics module may include at least one functional element, the at least one functional element being provided within the housing on the inside of the carrier and supporting the carrier, in particular wherein the sensor and/or the main board constitutes one of the at least one functional element.

In this context the inside is the side of the carrier facing away from the contact element. More specifically, no part of the housing is provided between the carrier and the functional element.

In one design variant, the housing has an opposing wall that is opposite the outer wall and against which the functional element rests, as a result of which the contact element is securely and permanently fixed in place.

For example, the outer wall is a base of the housing and/or the opposing wall is a cover of the housing, so that the electronics module can be simply electrically contacted via the base, in particular by means of the at least one contact element.

In one embodiment of the disclosure, the electronics module includes at least two contact elements and at least two functional elements, the carrier being supported by one of the at least two functional elements at one of the at least two contact elements and by another of the at least two functional elements at another of the at least two contact elements. In this way, the electronics module can be further reduced in size.

The object is further achieved by a wearable sensor system including a piece of clothing and an electronics module as described above. The piece of clothing includes a glove and a holder attached to the glove for a repeatable, tool-free attachment of the electronics module to the glove.

In the context of this disclosure, a wearable sensor system is understood to mean that the sensor system can be worn on the user's body like a piece of clothing.

The features and advantages discussed with regard to the electronics module correspondingly apply to the wearable sensor system and vice versa.

The glove may be a full glove or some other piece of hand clothing, such as a fingerstall or a wrist warmer.

For example, the holder includes at least one mating contact which electrically contacts the at least one contact element of the electronics module when the electronics module is inserted in the holder, the piece of clothing including a functional component attached at least to and/or in the glove, in particular a trigger, which is electrically connected to the mating contact. The functional component allows the electronics module to be extended in a simple manner.

### DESCRIPTION OF THE DRAWINGS

Further features and advantages of the disclosure will be apparent from the description below and from the accompanying drawings, to which reference is made and in which:

FIG. 1 schematically shows a wearable sensor system according to the disclosure;

FIG. 2 shows a perspective view of an electronics module according to the disclosure of the sensor system according to FIG. 1;

FIG. 3 shows a sectional view of the electronics module according to FIG. 2 along the line III-III of FIG. 4;

FIG. 4 schematically shows a bottom view of the upper part of the housing of the electronics module according to FIG. 2, with functional elements inserted;

FIG. 5 shows a schematic top view of a lower part of the housing of the electronics module according to FIG. 2; and

FIG. 6 shows a perspective view of a carrier of the electronics module according to FIG. 2.

### DETAILED DESCRIPTION

In general, the term “at least one of A and B” means “A and/or B”, namely “A” alone, “B” alone or “A and B”. The same holds true for listings with more than two items.

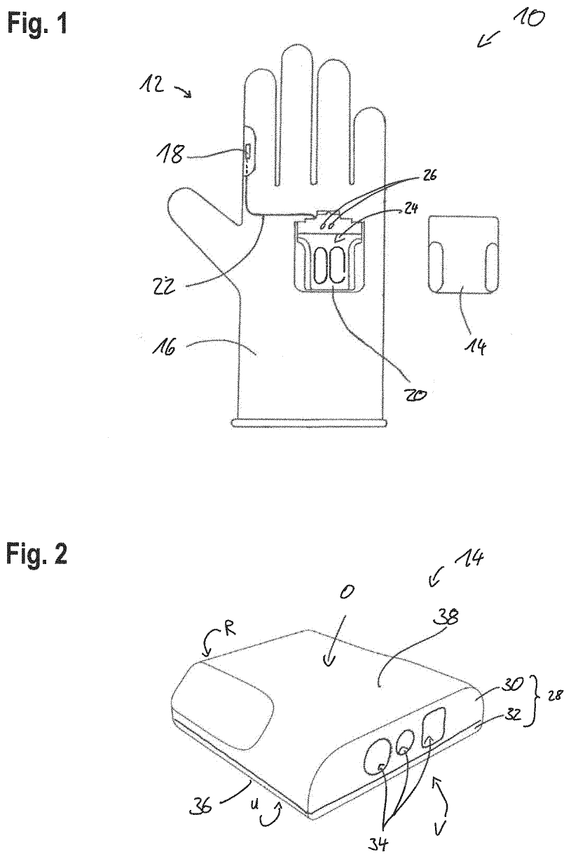

FIG. 1 illustrates a wearable sensor system 10 with a piece of clothing 12 and an electronics module 14.

The piece of clothing 12 includes a glove 16, a functional component 18, for example a push button, a holder 20 and a cable 22.

The glove 16 may be in the form of a full glove, as shown in FIG. 1, or a different piece of hand clothing, such as a fingerstall or a wrist warmer.

The holder 20 has a retainer 24 for the electronics module 14 and mating contacts 26, which are connected to the functional component 18 by means of the cable 22. In the exemplary embodiment shown, two mating contacts 26 are provided.

The functional component 18 is, for example, a trigger which short-circuits the mating contacts 26.

For example, the glove is an elastic and tight-fitting work glove that meets occupational safety requirements, e.g. for assembly operations.

As can also be seen in FIG. 2, the electronics module 14 is configured to be complementary to the retainer 24, so that the electronics module 14 can be fastened in the retainer 24 repeatably and without tools and can be removed from the retainer 24 again.

The electronics module 14 includes a housing 28, which has an upper part 30 and a lower part 32.

Due to the upper part 30 and the lower part 32, the housing 28 has a plurality of outer walls.

Furthermore, the housing 28 has a front side V, a rear side R, a lower side U and an upper side O.

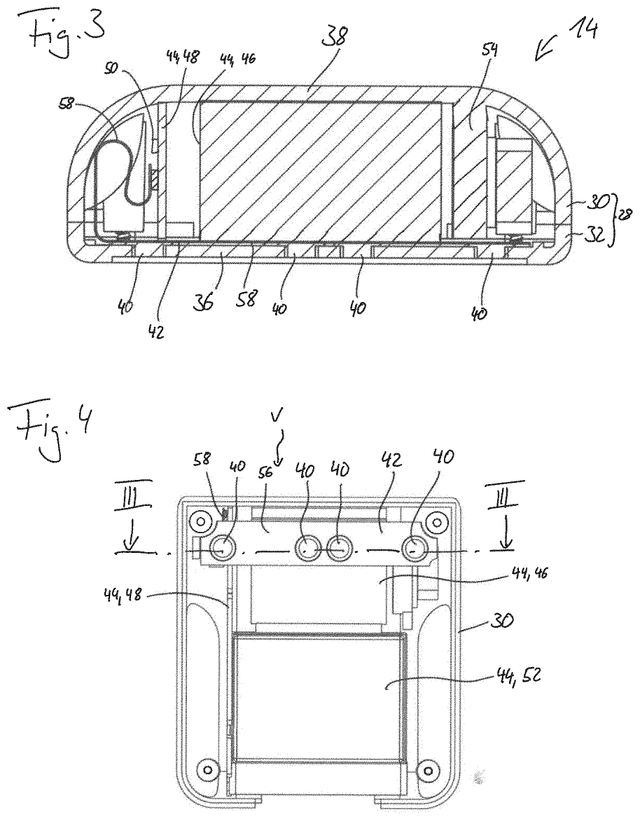

In the exemplary embodiment shown, a plurality of recesses 34 are provided on the front side V, in particular in the upper part 30, which serve the function of functional elements 44 (FIG. 3) of the electronics module 14.

The recesses 34 may be formed by windows and/or transparent housing sections, for example.

On the lower side U, an outer wall constitutes a base 36, which is provided correspondingly in the lower part 32 or as part of the lower part 32.

In the exemplary embodiment shown, the corresponding opposing wall of this outer wall, i.e. of the base 36, is a cover 38 of the housing 28, which is formed in the upper part 30 or as part of the upper part 30 and constitutes the upper side O of the housing 28 and thus of the electronics module 14.

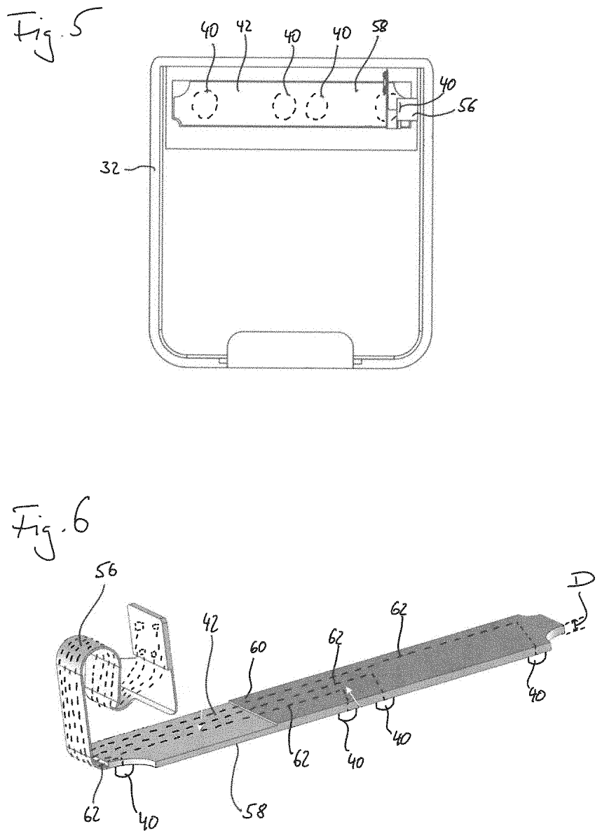

FIGS. 3, 4 and 5 show the electronics module 14 in detail in section and in an open position, respectively.

In addition to the housing 28, the electronics module 14 includes four contact elements 40, a carrier 42 for the contact elements 40, and a plurality of functional elements 44.

The carrier 42 and the functional elements 44 are provided inside the housing 28.

The contact elements 40 extend through an outer wall of the housing 28, in the exemplary embodiment shown through the base 36.

For simplification, it will only be described in the following that the contact elements extend through the base 36, it being understood that the contact elements 40 could also extend through any other outer wall of the housing 28.

In the exemplary embodiment shown, one of the functional elements 44 is a sensor 46, more precisely a bar code scanner with a camera. The camera of the bar code scanner is arranged such that it can detect the area in front of the front side V of the electronics module 14 through one of the recesses 34.

Of course, other sensors may also be provided, e.g. an RFID reader.

The bar code scanner can, for example, detect and evaluate one-dimensional and/or two-dimensional codes that are two-colored or multi-colored.

A further functional element 44 of the electronics module 14 is a main board 48, on which a controller 50 for the entire electronics module 14 is provided, among other things.

The main board 48 is arranged perpendicular to the base 36 and the cover 38.

In the exemplary embodiment, a battery or accumulator 52 is illustrated as a further functional element 44.

The sensor 46 and also the accumulator 52 are electrically connected to the main board 48.

Further functional elements 44, i.e. prefabricated electrical components may also be provided inside the housing 28. In the context of this disclosure, a functional element 44 is understood to mean a component other than a non-electronic and merely supporting component such as a wall or strut.

As can be clearly seen in FIG. 3, the functional elements 44, in particular the sensor 48 and the main board 48, rest against the cover 38 of the housing 28 and extend toward the base 36.

Here, the functional elements 44 are each located in the area of at least one of the contact elements 40.

The area of a contact element 40 is the area of the electronics module 14 which is located in an imaginary axial extension, e.g. perpendicular to the outer wall, of the corresponding contact element 40.

In the exemplary embodiment shown, the main board 48 is located in the area of the left contact element 40 and the sensor 46 extends over the area of the two middle contact elements 40.

In the area of the right contact element 40, a rib 54 of the housing 28 is provided, which also extends from the cover 38 toward the base 36.

FIG. 6 illustrates the carrier 42 in detail, which extends parallel to the front side V. The carrier 42 is arranged between the base 36 or the contact elements 40, on the one hand, and the functional elements 44, on the other hand.

In the exemplary embodiment shown, the carrier 42 is a rigid-flex printed circuit board and has a line section 56 and a holding section 58.

The line section 56 is constructed together with the holding section 58, that is, in one piece, and continues into it.

The carrier 42 has a thickness D of less than 1 mm in the holding section 58 and is even thinner in the line section 56 in particular. This gives the carrier 42 a flexible design.

The thickness D of the carrier 42 in the holding section 58 is, e.g., less than 0.8 mm, in particular less than 0.6 mm.

In the line section 56, the thickness of the carrier 42 is, e.g., between 0.3 and 0.5 mm, in particular 0.45 mm.

The holding section 58 is at least partly formed from an FR-4 material and may have a reinforcement layer 60 at least in sections. Here, the thickness of the reinforcement layer 60 is also included in the thickness D of the carrier 42 just mentioned above.

The reinforcement layer 60 may also be made of an FR-4 material.

FR-4 material is a commonly used material for the manufacture of printed circuit boards and is therefore known per se.

In the exemplary embodiment shown, the reinforcement layer 60 is provided in the region of the sensor 46, but not in the region of the main board 48.

The carrier 42 rests, in sections, against the functional elements 44, more precisely against the sensor 46 and the main board 48 as well as the rib 54, by its inner side, i.e. the side facing the inside of the housing 28 and thus the functional elements 44. Thereby, the carrier 42 is supported against the functional elements 44 and the rib 54, so that the carrier 42 is fixed in place in relation to the cover 38, which constitutes the opposing wall in the exemplary embodiment shown.

Here, no section of the housing 28 or any otherwise reinforcing component connected to the housing 28 is provided between the carrier 42 and the functional elements 44. In particular, the carrier 42 rests directly against the functional elements 44.

For example, the reinforcement layer 60 serves to compensate for differences in size between the functional elements 44.

The line section 56, for example, does not have an FR-4 material, but is formed, for example, from at least one film, such as a polyimide film, among other things.

Electrical conductors 62 are formed on or within the carrier 42, which are electrically connected to the contact elements 40 and extend along the entire carrier 42.

The line section 56 of the carrier 42 is attached to the main board 48, so that an electrical connection between the contact elements 40 and the main board 48 is provided by means of the electrical conductors 62 of the carrier 42.

For example, no further electrical components, in particular no active electrical components, are provided on the carrier 42 apart from the conductors 62.

The holding section 58 of the carrier is provided between the functional elements 44, on the one hand, and the base 36 or the contact elements 40, on the other.

The contact elements 40 are attached directly to the carrier 42, e.g. by gluing.

More precisely, the contact elements 40 are provided on the outside, i.e. on the side of the carrier 42 or of the holding section 58 facing the base 36. Gluing is effected, for example, by means of adhesive pads.

When the electronics module 14 is inserted into the retainer 24 of the holder 20, some of the contact elements 40—in this case the two middle contact elements 40—are brought into electrical contact with the mating contacts 26 of the holder 20. In this way, the functional component 18 can be used as a further component of the electronics module 14.

Here, the contact elements 40 are acted upon by a force in their axial direction, in this case towards the cover 38, by the mating contacts 26.

Owing to the small thickness D of the carrier 42, in particular of the holding section 58, the carrier 42 is so flexible that it cannot completely and in particular not permanently support the contact elements 40 when they are acted upon by a force in their axial direction, i.e. in a direction toward the cover 38.

However, since the carrier 42 is supported at the functional elements 44 and also at the rib 54 in the areas of the contact elements 40, the contact elements 40 are permanently fixed in place with respect to the cover 38, so that the flexible design of the carrier 42 is possible without any loss of functionality or service life.

This allows the total overall height of the electronics module 14 to be reduced.

The overall height of the electronics module 14 is thus merely made up of the sum of the thickness of the cover 38, the thickness of the functional element 44, the thickness D of the carrier 42 and the length of the contact elements 40.

1. An electronics module for a wearable sensor system, comprising a housing that includes at least one outer wall, at least one contact element, and a carrier for the at least one contact element,
wherein the contact element extends through the at least one outer wall and is attached to the carrier, and
wherein the carrier includes at least one electrical conductor for an electrical connection of the contact element and is designed to be flexible.

2. The electronics module according to claim 1, wherein the carrier is a rigid-flex printed circuit board.

3. The electronics module according to claim 1, wherein the carrier includes at least one line section and a holding section, the at least one contact element being attached to the holding section.

4. The electronics module according to claim 3, wherein the holding section is at least partly formed from an FR-4 material.

5. The electronics module according to claim 3, wherein the carrier includes a reinforcement layer in at least parts of the holding section

6. The electronics module according to claim 1, wherein the thickness of the carrier is equal to or less than 0.8 mm.

7. The electronics module according to claim 6, wherein the thickness of the holding section of the carrier is equal to or less than 0.8 mm.

8. The electronics module according to claim 1, wherein the electronics module includes at least one of at least one sensor and a main board.

9. The electronics module according to claim 8, wherein the at least one sensor is a camera.

10. The electronics module according to claim 9, wherein the main board is arranged perpendicular to the outer wall.

11. The electronics module according to claim 10, wherein the carrier is attached to the main board.

12. The electronics module according to claim 9, wherein the sensor is at least one of provided at least partly inside the housing and electrically connected to the main board.

13. The electronics module according to claim 1, wherein the electronics module includes at least one functional element, the at least one functional element being provided within the housing on the inside of the carrier and supporting the carrier.

14. The electronics module according to claim 13 wherein at least one of the sensor and the main board constitutes one of the at least one functional element.

15. The electronics module according to claim 1, wherein the housing has an opposing wall that is opposite the outer wall and against which the functional element rests.

16. The electronics module according to claim 15, wherein at least one of the outer wall is a base of the housing and the opposing wall is a cover of the housing.

17. The electronics module according to claim 1, wherein the electronics module includes at least two contact elements and at least two functional elements, the carrier being supported by one of the at least two functional elements at one of the at least two contact elements and by another of the at least two functional elements at another of the at least two contact elements.

18. A wearable sensor system comprising a piece of clothing and an electronics module according to claim 1,
the electronics module comprising a housing that includes at least one outer wall, at least one contact element, and a carrier for the at least one contact element,
wherein the contact element extends through the at least one outer wall and is attached to the carrier, and
wherein the carrier includes at least one electrical conductor for an electrical connection of the contact element and is designed to be flexible,
wherein the piece of clothing includes a glove and a holder attached to the glove for a repeatable, tool-free attachment of the electronics module to the glove.

19. The wearable sensor system according to claim 18, wherein the holder includes at least one mating contact which electrically contacts the at least one contact element of the electronics module when the electronics module is inserted in the holder, the piece of clothing including a functional component being at least one of attached to the glove and in the glove, which is electrically connected to the mating contact.

20. The wearable sensor system according to claim 19, wherein the functional component is a trigger.
